(19) United States
(12) Patent Application Publication
(10) Pub. No.: US 2025/0376708 A1
(43) Pub. Date: Dec. 11, 2025

(54) Method for mRNA Capping

(71) Applicant: Institute of Process Engineering, Chinese Academy of Sciences, Beijing (CN)

(72) Inventors: Songping Zhang, Beijing (CN); Zhiguo Su, Beijing (CN); Shiyi Che, Beijing (CN); Zhengjun Li, Beijing (CN); Xuan Lin, Beijing (CN); Xue Feng, Beijing (CN)

(21) Appl. No.: 19/170,076

(22) Filed: Apr. 4, 2025

Related U.S. Application Data
(63) Continuation of application No. PCT/CN2023/136692, filed on Dec. 6, 2023.

(30) Foreign Application Priority Data
Dec. 22, 2022 (CN) 202211659059.2

(51) Int. Cl. C12P 19/34

(52) U.S. Cl. CPC C12P 19/34

(57) ABSTRACT

Disclosed in the present application is a method for mRNA capping. The method comprises connecting mRNA to a stationary phase and carrying out a capping reaction to obtain a capped mRNA. The present application creatively establishes an immobilized mRNA-based capping reaction-separation coupling strategy and provides an efficient, simple, convenient, and low-cost capping method, and the capping operation can be carried out intermittently and can also be continuously carried out on a column. The present application provides a research basis for the development and application of a novel mRNA production process.

### FIELD

This application belongs to the field of nucleic acid technology and specifically relates to method for mRNA capping.

### BACKGROUND

Natural mRNA has a single-stranded structure, consisting of a 5′-methylguanosine cap (5′-cap), a 3′-polyadenylate tail (3′-polyA), and an intermediate protein translational and untranslated region. The 5′-cap can eliminate the free phosphate groups in the mRNA sequence, preventing ribonucleases and exonucleases from digesting the mRNA, thereby significantly enhancing the stability of the mRNA. Meanwhile, the 5′-cap can facilitate the recognition of mRNA by ribosomes and improve the translation efficiency by binding to the eukaryotic translation initiation factor 4E (eIF4E). According to the degree of methylation in the 5′-cap, there are three types of caps in mRNA, namely cap-0 (m⁷GpppN), cap-1 (m⁷GpppNm), and cap-2 (m⁷GpppNmNm). Pattern recognition receptors (PRRs) in immune cells can recognize uncapped mRNA or mRNA with a cap-O cap and inhibit their translation. However, mRNA with cap-1 and cap-2 cap structures can still be translated after being recognized. Currently, the mRNA used for therapeutic purposes is all obtained by in vitro transcription, that is, mRNA is transcribed from DNA templates under the action of RNA polymerases. To improve the structural stability and translation efficiency of the post-transcriptional mRNA, it is necessary to further cap it.

Currently, the capping methods for mRNA include enzymatic capping, co-transcriptional capping, and chemical capping. Among them, the most commonly used one is enzymatic capping. That is, the cap-O cap is first produced by the vaccinia virus capping enzyme with three enzymatic activities. The specific reaction mechanism of enzymatic capping is as follows: The mRNA loses a phosphate group at the 5′ end under the action of RNA triphosphatase. Then, the guanosine transferase adds the guanosine monophosphate (GMP) structure from the guanosine triphosphate (GTP) molecule to the mRNA that has lost a phosphate group. Finally, a methyl group is added to the N7 position of the guanine structure through the catalysis of guanosine methyltransferase, thus generating the mRNA with the cap-0 cap structure. In addition, under the action of 2-0′ methyltransferase, the mRNA with the cap-0 cap structure can add a methyl group at the 2-0′ end to generate the mRNA with the cap-1 cap structure. However, the enzymatic capping method has many reaction components. It requires a five-step operation of “adsorption-elution-capping-re-adsorption-re-elution” to obtain the purified capped mRNA. There are many separation steps, high cost, low mRNA yield, large loss, and it is prone to degradation.

WO2016193226A1 discloses a method for capping RNA using immobilized capping enzymes. In this method, the stationary phases of both the vaccinia virus capping enzyme and the 2′-O-methyltransferase are epoxy methacrylate magnetic beads. After being activated by epoxy groups, this stationary phase can form thiol groups with the cysteine residues of the enzyme molecules, thereby immobilizing the two capping enzymes. The immobilization conditions are as follows: 100 mM potassium phosphate, 500 mM sodium chloride, 1 mM ethylenediaminetetraacetic acid (pH=7.5). After being immobilized at 20° C. for 60 minutes, the protease concentration in the supernatant was measured to be basically zero, indicating that this method has a relatively high efficiency of immobilizing capping enzymes. Through analysis by a high-performance liquid chromatography column, the capping enzymes have relatively high activity and stability after being immobilized by this method. However, this method still requires first using a medium to adsorb and elute the mRNA for separation, then combining it with the immobilized capping enzymes to carry out the capping reaction, and subsequently still needs to separate the mRNA from other components except the capping enzymes, with problems such as mRNA loss existing.

CN112626177A discloses a method for rapidly and quantitatively detecting the capping efficiency of RNA. The method includes the following steps: S1. Synthesize uncapped RNA by in vitro transcription and remove the template DNA; S2. Perform capping treatment on the RNA; S3. Carry out monophosphorylation treatment on the RNA obtained from steps S1 and S2: First, use alkaline phosphatase for dephosphorylation, and after purifying the RNA, use polynucleotide kinase to add a monophosphate group to the 5′ end of the RNA; S4. Remove the RNA after monophosphorylation treatment using monophosphatase, and set up a control group without treatment by monophosphatase; S5. Conduct gel electrophoresis detection, quantitatively measure the band brightness of the RNA treated with monophosphatase (denoted as n) and the band brightness of the RNA in the control group (denoted as N), and calculate the capping efficiency of the RNA as: (n/N)×100%. This method can rapidly and quantitatively detect the capping efficiency of RNA. It is simple and fast to operate, with effective and accurate results. It requires a small amount of samples and has no special requirements for RNA types, lengths, etc., thus having a wide range of applications. However, this method has problems such as many separation steps before and after capping, the purification process is likely to cause quality loss of mRNA, the process takes a long time, and it is prone to cause the degradation of mRNA.

In conclusion, the current mRNA capping methods have problems such as numerous steps, high cost, low mRNA yield, large losses, and easy degradation. How to provide a simpler, faster, and lower-cost mRNA capping method has become one of the urgent problems to be solved in the field of nucleic acid technology at present.

### SUMMARY

This application provides a method for capping mRNA, which solves the problems existing in the current mRNA capping methods, such as numerous steps, high cost, low mRNA yield, large losses, and easy degradation, thus realizing convenient, efficient, and low-cost capping of mRNA.

This application provides a method for capping mRNA. The method includes: connecting the mRNA to the stationary phase and then conducting a capping reaction to obtain the capped mRNA.

This application creatively establishes a capping reaction-separation coupling strategy based on immobilized mRNA, providing an efficient, simple, and low-cost capping method. The capping operation can be carried out either intermittently or continuously on a column, providing a research basis for the development and application of new mRNA production processes.

In some embodiments, the mRNA is obtained by in vitro transcription.

In some embodiments, the reaction system for in vitro transcription includes RNA polymerase and a DNA template.

In some embodiments, the RNA polymerase includes one or a combination of at least two of T7 RNA polymerase, SP6 RNA polymerase, or T3 RNA polymerase.

In some embodiments, the DNA template is a DNA sequence with the function of encoding proteins, including one or a combination of at least two of linearized plasmids, PCR products, and synthesized DNA fragments.

In some embodiments, the mRNA is connected to the stationary phase through non-covalent interactions.

In some embodiments, the non-covalent interactions include one or a combination of at least two of hydrophobic interactions, electrostatic interactions, and affinity interactions.

In some embodiments, the stationary phase includes a solid material with a ligand capable of binding to mRNA on its surface.

In some embodiments, the ligand capable of binding to mRNA includes one or a combination of at least two of hydrophobic ligands, cationic ligands, and affinity ligands.

In some embodiments, the capping reaction involves contacting the mRNA with an mRNA capping enzyme to add a cap structure at the 5′ end of the mRNA.

In some embodiments, the mRNA capping enzymes include mRNA capping enzyme 1 and mRNA capping enzyme 2.

In some embodiments, the mRNA capping enzyme 1 includes a heterodimer composed of two subunits, D1 and D12, and more preferably, it is a capping enzyme derived from the vaccinia virus.

In some embodiments, the mRNA capping enzyme 2 includes 2-O′ methyltransferase.

In some embodiments, the cap structure at the 5′ end of the capped mRNA includes one of cap-0 (m⁷GpppN), cap-1 (m⁷GpppNm), or cap-2 (m⁷GpppNmNm).

In some embodiments, the method for capping mRNA includes the following steps:
(1) connecting the mRNA to the stationary phase;
(2) conducting a capping reaction on the mRNA connected to the stationary phase;
(3) washing away the unreacted components; and
(4) dissociating the capped mRNA from the stationary phase.

The schematic diagram of the mRNA capping process is shown in FIG. 1.

In some embodiments, the method for connecting the mRNA to the stationary phase includes: mixing the mRNA with the binding buffer, heating it and then cooling it on an ice bath, and subsequently adding the mRNA to the stationary phase that has been pre-equilibrated with the binding buffer for binding.

In some embodiments, the binding buffer includes one or a combination of at least two of Tris-HCl, sodium chloride, ethylenediaminetetraacetic acid (EDTA), and ammonium sulfate.

In some embodiments, the concentration of Tris-HCl is 0-50 mM.

The specific point values within the range of 0-50 mentioned above can be selected from 0, 3, 5, 7, 10, 20, 30, 40, 45, 48, 49, or 50, etc.

In some embodiments, the concentration of sodium chloride is 0-1 M.

The specific point values within the range of 0-1 mentioned above can be selected from 0, 0.1, 0.2, 0.3, 0.4, 0.5, 0.6, 0.7, 0.8, 0.9, or 1, etc.

In some embodiments, the concentration of ethylenediaminetetraacetic acid (EDTA) is 0-1 mM.

The specific point values within the range of 0-1 mentioned above can be selected from 0, 0.1, 0.2, 0.3, 0.4, 0.5, 0.6, 0.7, 0.8, 0.9, or 1, etc.

In some embodiments, the concentration of ammonium sulfate is 0-1 M.

The specific point values within the range of 0-1 mentioned above can be selected from 0, 0.1, 0.2, 0.3, 0.4, 0.5, 0.6, 0.7, 0.8, 0.9, or 1, etc.

In some embodiments, the heating temperature is 35-65° C.

The specific point values within the range of 35-65 mentioned above can be selected from 35, 36, 40, 45, 50, 55, 60, 62, 63, 64, or 65, etc.

In some embodiments, the heating time is 5-10 minutes.

The specific point values within the range of 5-10 mentioned above can be selected from 5, 6, 7, 8, 9, or 10, etc.

In some embodiments, the binding ratio of mRNA to the stationary phase is 0.2-8 μg/μL, preferably 1-4 μg/μL.

The specific point values within the range of 0.2-8 mentioned above can be selected from 0.2, 2, 3, 4, 5, 6, 7, or 8, etc.

The specific point values within the range of 1-4 mentioned above can be selected from 1, 2, 3, or 4, etc.

In some embodiments, the binding temperature is 25-65° C.

The specific point values within the range of 25-65 mentioned above can be selected from 25, 36, 40, 45, 50, 55, 60, 62, 63, 64, or 65, etc.

In some embodiments, the binding time is 5-120 minutes.

The specific point values within the range of 5-120 mentioned above can be selected from 5, 30, 40, 45, 50, 55, 60, 80, 90, 110, or 120, etc.

In some embodiments, the capping reaction described in step (2) includes: adding the capping buffer containing guanosine triphosphate and S-adenosylmethionine to the stationary phase with adsorbed mRNA, and then adding mRNA capping enzyme 1 and mRNA capping enzyme 2 to conduct the capping reaction.

In some embodiments, the concentration of guanosine triphosphate is 0.1-1 mM.

The specific point values within the range of 0.1-1 mentioned above can be selected from 0.1, 0.2, 0.3, 0.4, 0.5, 0.6, 0.7, 0.8, 0.9, or 1, etc.

In some embodiments, the concentration of S-adenosylmethionine is 0.1-1 mM.

The specific point values within the range of 0.1-1 mentioned above can be selected from 0.1, 0.2, 0.3, 0.4, 0.5, 0.6, 0.7, 0.8, 0.9, or 1, etc.

In some embodiments, the capping buffer further includes one or a combination of at least two of Tris-HCl, potassium chloride, magnesium chloride, and dithiothreitol.

In some embodiments, the concentration of Tris-HCl is 0-50 mM.

The specific point values within the range of 0-50 mentioned above can be selected from 0, 10, 15, 20, 25, 30, 35, 40, 45, or 50, etc.

In some embodiments, the concentration of potassium chloride is 0-10 mM.

The specific point values within the range of 0-10 mentioned above can be selected from 0, 1, 2, 3, 4, 5, 6, 7, 8, 9, or 10, etc.

In some embodiments, the concentration of magnesium chloride is 0-1 mM.

The specific point values within the range of 0-1 mentioned above can be selected from 0, 0.1, 0.2, 0.3, 0.4, 0.5, 0.6, 0.7, 0.8, 0.9, or 1, etc.

In some embodiments, the concentration of dithiothreitol is 0-1 mM.

The specific point values within the range of 0-1 mentioned above can be selected from 0, 0.1, 0.2, 0.3, 0.4, 0.5, 0.6, 0.7, 0.8, 0.9, or 1, etc.

In some embodiments, the temperature of the capping reaction is 25-65° C.

The specific point values within the range of 25-65 mentioned above can be selected from 25, 36, 40, 45, 50, 55, 60, 62, 63, 64, or 65, etc.

In some embodiments, the time of the capping reaction is 5-60 minutes.

The specific point values within the range of 5-60 mentioned above can be selected from 5, 30, 40, 45, 50, 55, or 60, etc.

In some embodiments, the method for dissociation described in step (4) includes adding the elution buffer to the stationary phase with the capped mRNA attached, performing elution, and collecting the capped mRNA.

In some embodiments, the elution buffer includes one or a combination of Tris-HCl, sodium chloride, and ethylenediaminetetraacetic acid.

In some embodiments, the concentration of Tris-HCl is 0-50 mM.

The specific point values within the range of 0-50 mentioned above can be selected from 0, 10, 15, 20, 25, 30, 35, 40, 45, or 50, etc.

In some embodiments, the concentration of sodium chloride is 0-1 M.

The specific point values within the range of 0-1 mentioned above can be selected from 0, 0.1, 0.2, 0.3, 0.4, 0.5, 0.6, 0.7, 0.8, 0.9, or 1, etc.

In some embodiments, the concentration of ethylenediaminetetraacetic acid (EDTA) is 0
1 mM.

The specific point values within the range of 0-1 mentioned above can be selected from 0, 0.1, 0.2, 0.3, 0.4, 0.5, 0.6, 0.7, 0.8, 0.9, or 1, etc.

In some embodiments, the temperature for the elution is 25-65° C.

The specific point values within the range of 25-65 mentioned above can be selected from 25, 36, 40, 45, 50, 55, 60, 62, 63, 64, or 65, etc.

In some embodiments, the time for the elution is 5-60 minutes.

The specific point values within the range of 5-60 mentioned above can be selected from 5, 30, 40, 45, 50, 55, or 60, etc.

Compared with the prior art, this application has the following beneficial effects:
(1) The step of denaturing pretreatment of mRNA before the capping reaction is reduced, making the operation more convenient.
(2) After the capping reaction, the separation of mRNA and other reaction components can be achieved simply by washing and rinsing operations, reducing the additional purification steps after the reaction and simplifying the production process of mRNA.
(3) Purified capped mRNA can be obtained through a one-step elution after the reaction, which improves the stability and yield of mRNA.

### BRIEF DESCRIPTION OF DRAWINGS

FIG. 1 is a schematic diagram of the process of capping immobilized mRNA.

FIG. 2 is the agarose gel electrophoresis diagrams of Example 1 and Comparative Example 1 before and after capping.

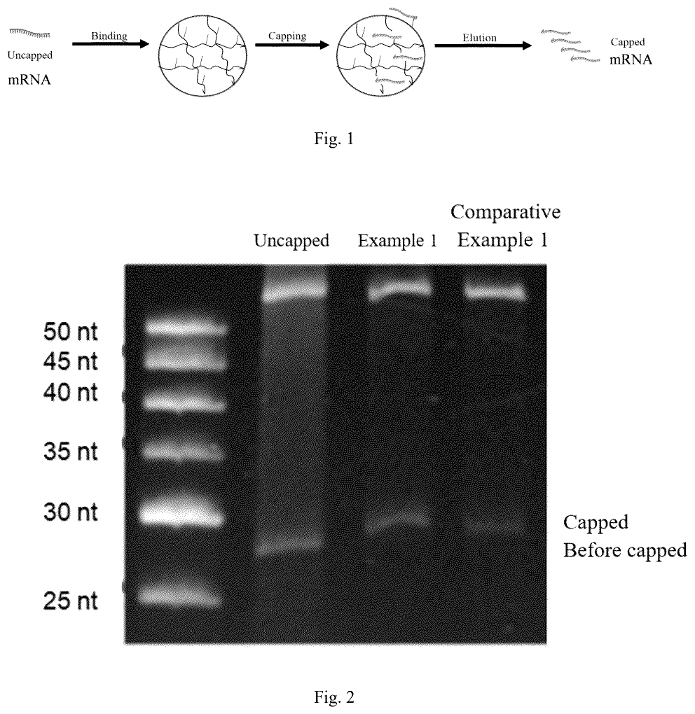

### DETAILED DESCRIPTION

To further elaborate on the technical means adopted in this application and their effects, the following provides a further explanation of this application in combination with examples and accompanying drawings. It can be understood that the specific implementation methods described here are only used to explain this application and not to limit it.

For those in the examples where specific techniques or conditions are not indicated, the techniques or conditions described in the literature in the relevant field shall be followed, or those stated in the product manuals shall be followed. For the reagents or instruments used where the manufacturers are not indicated, they are all conventional products that can be purchased through regular channels.

### EXAMPLE 1

A method for capping immobilized mRNA.

(1) Take 50 μg of in vitro transcribed mRNA expressing green fluorescent protein, heat it to denature at 65° C., and then immediately cool it down in an ice bath. Add 50 μL of Oligo dT medium, which has been equilibrated with the equilibration buffer (10 mM Tris-HCl+1 M NaCl+1 mM EDTA, pH=7.4), to the mRNA and allow them to bind at 25° C. for 10 minutes.

(2) Add the buffer containing 1 mM GTP and 1 mM SAM to the stationary phase with adsorbed mRNA, mix well, and then add capping enzyme 1 (vaccinia virus capping enzyme) and capping enzyme 2 (2-O′ methyltransferase) to conduct the capping reaction for 30 minutes at a capping temperature of 45° C. Centrifuge to collect the stationary phase with adsorbed capped mRNA.

(3) Wash to remove the unreacted components.

(4) Elute the capped mRNA adsorbed on the stationary phase with the elution buffer (10 mM Tris-HCl+1 mM EDTA, pH=7.4) at 25° C. for 30 minutes. Centrifuge to collect the dissociated mRNA.

### EXAMPLE 2

A method for capping immobilized mRNA.

(1) Take 150 μg of in vitro transcribed mRNA expressing green fluorescent protein, heat it to denature at 35° C., and then immediately cool it down in an ice bath. Add 50 μL of DEAE medium, which has been equilibrated with the equilibration buffer (10 mM Tris-HCl+1 mM EDTA, pH=7.4), to the mRNA and allow them to bind at 40° C. for 60 minutes.

(2) Add the buffer containing 0.5 mM GTP and 0.5 mM SAM to the stationary phase with adsorbed mRNA, mix well, and then add capping enzyme 1 (vaccinia virus capping enzyme) and capping enzyme 2 (2-O′ methyltransferase) to conduct the capping reaction for 60 minutes at a capping temperature of 65° C. Centrifuge to collect the stationary phase with adsorbed capped mRNA.

(3) Wash to remove the unreacted components.

(4) Elute the capped mRNA adsorbed on the stationary phase with the elution buffer (10 mM Tris-HCl+1M NaCl+1 mM EDTA, pH=7.4) at 40° C. for 10 minutes. Centrifuge to collect the dissociated mRNA.

### EXAMPLE 3

A method for capping immobilized mRNA.

(1) Take 400 μg of in vitro transcribed mRNA expressing green fluorescent protein, heat it to denature at 45° C., and then immediately cool it down in an ice bath. Add 100 μL of Butyl-FF medium, which has been equilibrated with the equilibration buffer (20 mM Tris-HCl+1 M ammonium sulfate+1 mM EDTA, pH=7.4), to the mRNA and allow them to bind at 65° C. for 120 minutes.

(2) Add the buffer containing 0.1 mM GTP and 0.1 mM SAM to the stationary phase with adsorbed mRNA, mix well, and then add capping enzyme 1 (vaccinia virus capping enzyme) and capping enzyme 2 (2-O′ methyltransferase) to conduct the capping reaction for 10 minutes at a capping temperature of 25° C. Centrifuge to collect the stationary phase with adsorbed capped mRNA.

(3) Wash to remove the unreacted components.

(4) Elute the capped mRNA adsorbed on the stationary phase with the elution buffer (20 mM Tris-HCl+1 mM EDTA, pH=7.4) at 65° C. for 30 minutes. Centrifuge to collect the dissociated mRNA.

### EXAMPLE 4

A method for capping immobilized mRNA.

(1) Take 100 μg of in vitro transcribed mRNA expressing green fluorescent protein (with a volume of 200 μL), heat it to denature at 65° C., and then immediately cool it down in an ice bath.

(2) Load 100 μL of Oligo dT medium into the chromatography column tube and equilibrate it with the equilibration buffer (10 mM Tris-HCl+1M NaCl+1 mM EDTA, pH=7.4).

(3) Inject the mRNA from (1) into the chromatography column with a retention time of 10 minutes. The column temperature and injection temperature are both 25° C.

(4) After the injection is completed, rinse the unbound components with 600 μL of binding buffer.

(5) Inject 300 μL of the buffer containing 1 mM GTP, 1 mM SAM, capping enzyme 1 (vaccinia virus capping enzyme), and capping enzyme 2 (2-O′ methyltransferase) into the chromatography column and allow the reaction to occur on the column for 30 minutes at a temperature of 45° C.

(6) After the capping reaction is completed, rinse the chromatography column with 700 μL of equilibration buffer to remove the unreacted components.

(7) Under the condition of 25° C., elute the capped mRNA adsorbed on the fixed phase with 400 μL of elution buffer (10 mM Tris-HCl+1 mM EDTA, pH=7.4) and collect the eluted mRNA.

### COMPARATIVE EXAMPLE 1

Enzymatic capping of free mRNA.

(1) Take 50 μg of in vitro transcribed mRNA expressing green fluorescent protein, heat it to denature at 65° C., and then immediately cool it down in an ice bath. Add 50 μL of Oligo dT medium, which has been equilibrated (the equilibration buffer is: 10 mM Tris-HCl+1M NaCl+1 mM EDTA, pH=7.4), to the mRNA and allow them to bind at 25° C. for 10 minutes. Collect the stationary phase with adsorbed mRNA and remove the unbound mRNA. Elute the adsorbed mRNA with the elution buffer and collect the eluted components.

(2) Exchange the buffer of the eluted mRNA to the equilibration buffer through an ultrafiltration membrane.

(3) Blend the mRNA after buffer exchange, 1 mM GTP, 1 mM SAM, capping enzyme 1 (vaccinia virus capping enzyme), and capping enzyme 2 (2-O′ methyltransferase) together to conduct the capping reaction for 30 minutes at a capping temperature of 45° C.

(4) Bind the mixture containing the capped mRNA to 50 μL of dT affinity resin again and allow them to bind at 25° C. for 10 minutes to remove the unbound components.

(5) Elute the adsorbed capped mRNA with the elution buffer (10 mM Tris-HCl+1 mM EDTA, pH=7.4) at 25° C. for 30 minutes and collect the eluted components.

### TESTING EXAMPLE 1

This testing example is used to detect the capping efficiency.

The mRNA was digested with ribozyme, and the 5′-end mRNA fragments after digestion were analyzed by denaturing polyacrylamide gel electrophoresis. The test results are shown in FIG. 2. The 5′-end mRNA fragments after digestion were analyzed using a DNA Pac RP chromatographic column (2.1×100 mm, Thermo Fisher, USA). The peak areas of the capped and uncapped components in the liquid chromatogram were integrated. The capping rate was calculated in the following way:

$$Capping rate = \frac{The peak area of capped mRNA}{The peak area of uncapped mRNA + The peak area of capped mRNA} * 100 \%$$

The capping effects of Example 1-4 and Comparative Example 1 were detected using this method. The capping rate were analyzed based on the peak areas, as shown in Table 1.

TABLE 1

| Capping method | Capping rate (%) |
|---|---|
| Comparative Example 1 | 99.88 |
| Example 1 | 99.70 |
| Example 2 | 99.16 |
| Example 3 | 99.25 |
| Example 4 | 99.31 |

As can be seen from the test data in Table 1, compared with the capping of free mRNA, the capping of immobilized mRNA has a capping rate of over 99% whether in batch operation or continuous operation on a column. This indicates that the method of capping immobilized mRNA in this application can cap mRNA with high efficiency. It can be operated either in batch mode or continuously on a column, thus providing a research basis for the development and application of new mRNA production processes.

### TESTING EXAMPLE 2

This testing example is for the comparison of mRNA yield, purity, steps used, and time consumption between Example 1-4 and Comparative Example 1 under the same capping conditions.

The specific methods are as follows:

(1) Use NanoDrop to measure the concentration of mRNA in the eluate. The calculation formula for calculating the mRNA yield is shown as follows:

$$mRNA yield = \frac{Mass of mRNA after elution}{Total amount of mRNA added} * 100 \%$$

The test results are shown in Table 2.

TABLE 2

| Capping method | Capping rate (%) |
|---|---|
| Example 1 | 84.59 |
| Example 2 | 81.44 |
| Example 3 | 82.06 |
| Example | 80.22 |
| Comparative Example 1 | 45.62 |

As can be seen from the test data in Table 2, compared with the capping of free mRNA, the capping of immobilized mRNA results in a higher final mRNA yield, indicating that the method of capping immobilized mRNA in this application improves the yield of mRNA production.

(2) Detect the purity of mRNA by liquid chromatography: Use a TSK 6000 chromatographic column (7.8×300 mm, TOSOH, Japan) to detect the mRNA after elution. Integrate the peak areas of the capped and uncapped components in the liquid chromatogram. The calculation method for its purity is:

$$Purity = \frac{Peak area of mRNA}{Peak area of mRNA + Peak area of impurities} * 100 \%$$

The results of mRNA purity are shown in Table 3.

TABLE 3

| Capping method | Purity of mRNA (%) |
|---|---|
| Example 1 | 99.27 |
| Example 2 | 98.33 |
| Example 3 | 97.56 |
| Example 4 | 99.16 |
| Comparative Example 1 | 93.87 |

As can be seen from the test data in Table 3, compared with the capping of free mRNA, the capping of immobilized mRNA results in a higher final mRNA purity, indicating that the method of capping immobilized mRNA in this application improves the purity of mRNA.

(3) Calculate the total capping time consumption by adding up the time consumed in each step of Example 1, Example 4 and Comparative Example 1. The results are shown in Table 4.

TABLE 4

| Comparative Example 1 | | | Example 1 | | | Example 4 | | |
|---|---|---|---|---|---|---|---|---|
| | | Yield of | | | Yield of | | | Yield of |
| | Time | each step | | Time | each step | | Time | each step |
| Step | (min) | (%) | Step | (min) | (%) | Step | (min) | (%) |
| Binding | 10 | 94.61 | Binding | 10 | 94.53 | Binding | 10 | 92.33 |
| Elution | 30 | 87.16 | Capping | 30 | | Capping | 30 | — |
| Ultrafiltration | 20 | 80.26 | Elution | 30 | 89.48 | Elution | 30 | 86.88 |
| Capping | 30 | | | | | | | |
| Binding | 30 | 83.93 | | | | | | |
| Elution | 30 | 82.12 | | | | | | |
| Total | Total | Total | Total of | Total | Total | Total of | Total | Total |
| of six | time: 150 | yield: 45.62 | three | time: 70 | yield: 84.59 | three | time: 70 | yield: 80.22 |
| steps | | | steps | | | steps | | |

As can be seen from the test data in Table 4, under the condition of the same mRNA mass, compared with the capping of free mRNA, the capping of immobilized mRNA has a higher mRNA yield, and the number of steps and time consumption are both half of those of free mRNA. This indicates that the method of capping immobilized mRNA in this application improves the yield of mRNA production, reduces the reaction steps, and simplifies the mRNA production process.

In conclusion, this application creatively establishes an efficient, simple, and low-cost capping method. The capping operation can be carried out either in batch mode or continuously on a column, providing a research basis for the development and application of new mRNA production processes.

The applicant declares that this application uses the above examples to illustrate the detailed methods of this application. However, this application is not limited to the above detailed methods, that is, it does not mean that this application must rely on the above detailed methods to be implemented. Those skilled in the relevant technical field should understand that any improvement to this application, the equivalent replacement of each raw material of the products of this application, the addition of auxiliary components, the selection of specific methods, etc., all fall within the protection scope and the disclosure scope of this application.

1. A method for capping mRNA, which comprises: connecting the mRNA to a stationary phase and then conducting a capping reaction to obtain a capped mRNA.

2. The method of capping mRNA according to claim 1, wherein the mRNA is obtained by in vitro transcription.

3. The method of capping mRNA according to claim 1, wherein a reaction system for in vitro transcription includes RNA polymerase and DNA template;
preferably, the RNA polymerase includes one or a combination of at least two of T7 RNA polymerase, SP6 RNA polymerase, or T3 RNA polymerase;
preferably, the DNA template is a DNA sequence with the function of encoding a protein and includes one or a combination of at least two of linearized plasmid, PCR product, and synthesized DNA fragment.

4. The method of capping mRNA according to claim 1, wherein the mRNA is connected to the stationary phase by non-covalent interaction;
preferably, the non-covalent interaction includes one or a combination of at least two of hydrophobic interaction, electrostatic interaction, and affinity interaction;
preferably, the stationary phase includes a solid material with a ligand capable of binding to mRNA modified on its surface;
preferably, the ligand capable of binding to mRNA includes one or a combination of at least two of hydrophobic ligand, cationic ligand, and affinity ligand.

5. The method of capping mRNA according to claim 1, wherein capping reaction comprises contacting the mRNA with an mRNA capping enzyme to add a cap structure at the 5′ end of the mRNA.

6. The method of capping mRNA according to claim 5, wherein the mRNA capping enzymes include mRNA capping enzyme 1 and mRNA capping enzyme 2;
preferably, the mRNA capping enzyme 1 comprises a heterodimer composed of two subunits, D1 and D12, and more preferably is the capping enzyme derived from vaccinia virus;
preferably, the mRNA capping enzyme 2 includes 2-O′-methyltransferase.

7. The method of capping mRNA according to claim 5, wherein the cap structure at the 5′ end of the capped mRNA includes one of cap-0 (m⁷GpppN), cap-1 (m⁷GpppNm), or cap-2 (m⁷GpppNmNm).

8. The method of capping mRNA according to claim 1, wherein the method for capping mRNA comprises the following steps:
(1) connecting the mRNA to a stationary phase;
(2) conducting a capping reaction to the mRNA connected to the stationary phase;
(3) washing away unreacted components; and
(4) dissociating capped mRNA from the stationary phase.

9. The method of capping mRNA according to claim 8, wherein the method for connecting the mRNA to the stationary phase comprises: mixing the mRNA with a binding buffer, heating and then cooling on ice bath, and then adding the mRNA to the stationary phase which has been pre-equilibrated with the binding buffer for binding.

10. The method of capping mRNA according to claim 9, wherein the binding buffer includes one or a combination of at least two of Tris-HCl, sodium chloride, ethylenediaminetetraacetic acid (EDTA), and ammonium sulfate;
preferably, a concentration of Tris-HCl is 0-50 mM;
preferably, a concentration of sodium chloride is 0-1 M;
preferably, a concentration of ethylenediaminetetraacetic acid (EDTA) is 0-1 mM;
preferably, a concentration of ammonium sulfate is 0-1 M.

11. The method of capping mRNA according to claim 9, wherein a heating temperature is 35-65° C.;
preferably, a heating time is 5-10 minutes;
preferably, a binding ratio of the mRNA to the stationary phase is 0.2-8 μg/μL, and more preferably 1-4 μg/μL;
preferably, a binding temperature is 25-65° C.;
preferably, a binding time is 5-120 minutes.

12. The method of capping mRNA according to claim 8, wherein the capping reaction in step (2) comprises: adding a capping buffer containing guanosine triphosphate and S-adenosylmethionine to the stationary phase on which the mRNA is adsorbed, and then adding mRNA capping enzyme 1 and mRNA capping enzyme 2 to conduct the capping reaction;
preferably, a concentration of guanosine triphosphate is 0.1-1 mM;
preferably, a concentration of S-adenosylmethionine is 0.1-1 mM;
preferably, the capping buffer further includes one or a combination of at least two of Tris-HCl, potassium chloride, magnesium chloride, and dithiothreitol;
preferably, a concentration of Tris-HCl is 0-50 mM;
preferably, a concentration of potassium chloride is 0-10 mM;
preferably, a concentration of magnesium chloride is 0-1 mM;
preferably, a concentration of dithiothreitol is 0-1 mM;
preferably, a temperature of the capping reaction is 25-65° C.;
preferably, a time of the capping reaction is 5-60 minutes.

13. The method of capping mRNA according to claim 8, wherein the dissociation in step (4) comprises adding the elution buffer to the stationary phase to which the capped mRNA is attached, performing elution, and collecting the capped mRNA;
preferably, the elution buffer includes one or a combination of several of Tris-HCl, sodium chloride, and ethylenediaminetetraacetic acid;
preferably, a concentration of the Tris-HCl is 0-50 mM;
preferably, a concentration of the sodium chloride is 0-1 M;
preferably, a concentration of the ethylenediaminetetraacetic acid is 0-1 mM;
preferably, a temperature of the elution is 25-65° C.;
preferably, a time of the elution is 5-60 minutes.
